(19) United States
(12) Patent Application Publication
(10) Pub. No.: US 2025/0257050 A1
(43) Pub. Date: Aug. 14, 2025

(54) PROCESS FOR THE PREPARATION OF VORASIDENIB

(71) Applicant: LES LABORATOIRES SERVIER, Suresnes Cedex (FR)

(72) Inventors: Bruno COMMARE, Canville-les-Deux-Eglises (FR); Philippe BESCOND, Saint-Vrain (FR); Samuel OGER, Fécamp (FR)

(21) Appl. No.: 19/048,269

(22) Filed: Feb. 7, 2025

(30) Foreign Application Priority Data
Feb. 9, 2024 (EP) 24315044.8

(51) Int. Cl. C07D 401/04; C07C 277/02

(52) U.S. Cl. CPC C07D 401/04; C07C 277/02

(57) ABSTRACT

Process for the preparation of vorasidenib using N,N′-bis[(2R)-1,1,1-trifluoropropan-2-yl]triimidodicarbonic diamide hydrochloride

### RELATED APPLICATIONS

This application claims a right of priority under 35 U.S.C. § 119(a) to European Patent Application EP 24315044.8 filed on Feb. 9, 2024.

### FIELD

The present invention relates to a process for the preparation of vorasidenib, an orally available, brain penetrant second-generation dual mutant isocitrate dehydrogenase 1 and 2 (mIDH1/2) inhibitor.

### BACKGROUND

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2,4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production.

The human IDH1 gene encodes a protein of 414 amino acids. The nucleotide and amino acid sequences for human IDH1 can be found as GenBank entries NM_005896.2 and NP_005887.2 respectively. The nucleotide and amino acid sequences for IDH1 are also described in, e.g., Nekrutenko et al., Mol. Biol. Evol. 15:1674-1684(1998); Geisbrecht et al., J. Biol. Chem. 274:30527-30533(1999); Wiemann et al., Genome Res. 11:422-435(2001); The MGC Project Team, Genome Res. 14:2121-2127(2004); Lubec et al., Submitted (DEC-2008) to UniProtKB; Kullmann et al., Submitted (JUN-1996) to the EMBL/GenBank/DDBJ databases; and Sjoeblom et al., Science 314:268-274(2006).

Non-mutant, e.g., wild type, IDH1 catalyzes the oxidative decarboxylation of isocitrate to α-ketoglutarate.

It has been discovered that mutations of IDH1 present in certain cancer cells result in a new ability of the enzyme to catalyze the NADPH-dependent reduction of α-ketoglutarate to R(−)-2-hydroxyglutarate (2HG). The production of 2HG is believed to contribute to the formation and progression of cancer (Dang, L et al., Nature 2009, 462:739-44).

IDH2 (isocitrate dehydrogenase 2 (NADP+), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Human IDH2 gene encodes a protein of 452 amino acids. The nucleotide and amino acid sequences for IDH2 can be found as GenBank entries NM_002168.2 and NP_002159.2 respectively. The nucleotide and amino acid sequence for human IDH2 are also described in, e.g., Huh et al., Submitted (NOV-1992) to the EMBL/GenBank/DDBJ databases; and The MGC Project Team, Genome Res. 14:2121-2127(2004).

Non-mutant, e.g., wild type, IDH2 catalyzes the oxidative decarboxylation of isocitrate to α-ketoglutarate (α-KG).

It has been discovered that mutations of IDH2 present in certain cancer cells result in a new ability of the enzyme to catalyze the NADPH-dependent reduction of α-ketoglutarate to R(−)-2-hydroxyglutarate (2HG). 2HG is not formed by wild-type IDH2. The production of 2HG is believed to contribute to the formation and progression of cancer (Dang, L et al, Nature 2009, 462:739-44).

Recurrent or progressive IDH-mutant gliomas are oligodendrogliomas and astrocytomas that harbor IDH1 or IDH2 gene mutation and have recurred or progressed after receiving standard of care therapy including surgery, radiation and/or chemotherapy.

Vorasidenib is an orally available, brain penetrant second-generation dual mutant isocitrate dehydrogenase 1 and 2 (mIDH1/2) inhibitor approved in the USA for the treatment of IDH-mutant gliomas.

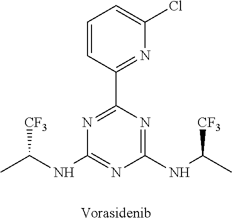

Vorasidenib or (6-(6-chloropyridin-2-yl)-N²,N⁴-bis((R)-1,1,1-trifluoropropan-2-yl)-1,3,5-triazine-2,4-diamine) is disclosed in PCT publication WO2015/003640.

PCT publication WO2015/003640 also discloses a lab-scale process for the preparation of vorasidenib starting from methyl 6-chloropyridine-2-carboxylate and according to the following reaction scheme:

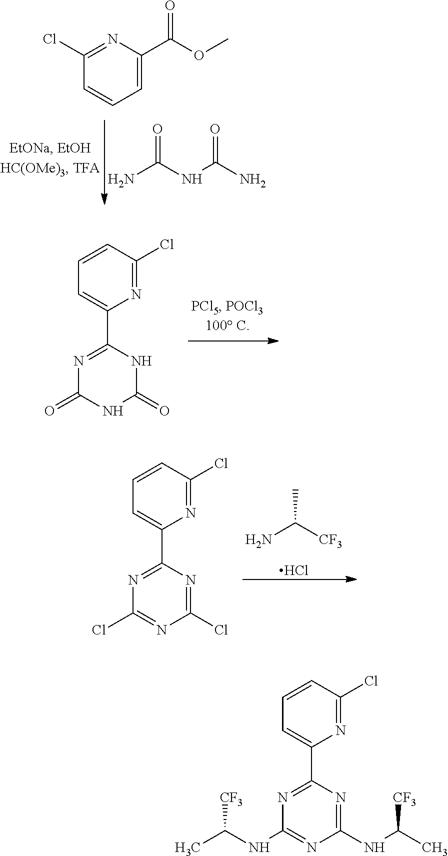

The 2,4-dichloro-6-(6-chloropyridin-2-yl)-1,3,5-triazine formed as an intermediate in this synthetic pathway is not very stable and should be stored in solution. In addition, use of POCl₃ as a reagent at industrial scale is difficult to manage, due to its high toxicity (it may be fatal by inhalation) and the potential for highly exothermic hydrolysis leading to the formation of phosphoric acid and hydrochloric acid. Its industrial use is strictly regulated in the EU.

PCT publication WO2015/003640 also discloses a process for preparation of disubstituted biguanides that are used as intermediates in the preparation of analogs of vorasidenib (refer to Scheme 19 for example). The operating conditions disclosed are implemented without solvent at 160° C. These harsh conditions are not suitable to be used in an industrial process, due to a high risk of explosion when warming large amounts of powders at such high temperature.

Consequently, there was a need to develop a safer process for the preparation of vorasidenib that may be used to produce this molecule at an industrial scale.

### SUMMARY

The present disclosure relates to a process for the preparation of vorasidenib of formula (I)

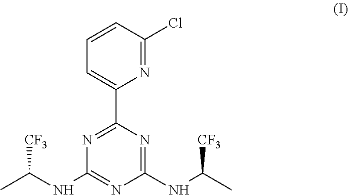

comprising reacting (R)-1,1,1-trifluoropropan-2-amine hydrochloride of formula (II):

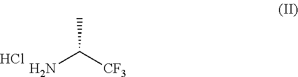

with sodium dicyanamide,
to yield N,N′-bis[(2R)-1,1,1-trifluoropropan-2-yl]triimidodicarbonic diamide hydrochloride of formula (III)

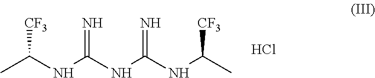 [SMILES: C[C@@H](NC(=N)NC(=N)N[C@H](C)C(F)(F)F)C(F)(F)F ; Cl]

followed by reacting the compound of formula (III) with methyl 6-chloropyridine-2-carboxylate. to yield vorasidenib.

Vorasidenib free form may be prepared by the new process and then transformed into the hemicitric acid hemihydrate cocrystal.

References made in this patent application to “hemicitric acid hemihydrate cocrystal” refer to hemicitric acid hemihydrate cocrystal Type A as described in WO 2019/090059.

The present disclosure also relates to the use of N,N′-bis[(2R)-1,1,1-trifluoropropan-2-yl]triimidodicarbonic diamide hydrochloride of formula (III) as an intermediate compound for the preparation of vorasidenib.

### BRIEF DESCRIPTION OF THE DRAWINGS

FIG. 1 depicts the 1H NMR spectrum of N,N′-bis[(2R)-1,1,1-trifluoropropan-2-yl]triimidodicarbonic diamide hydrochloride of formula (III).

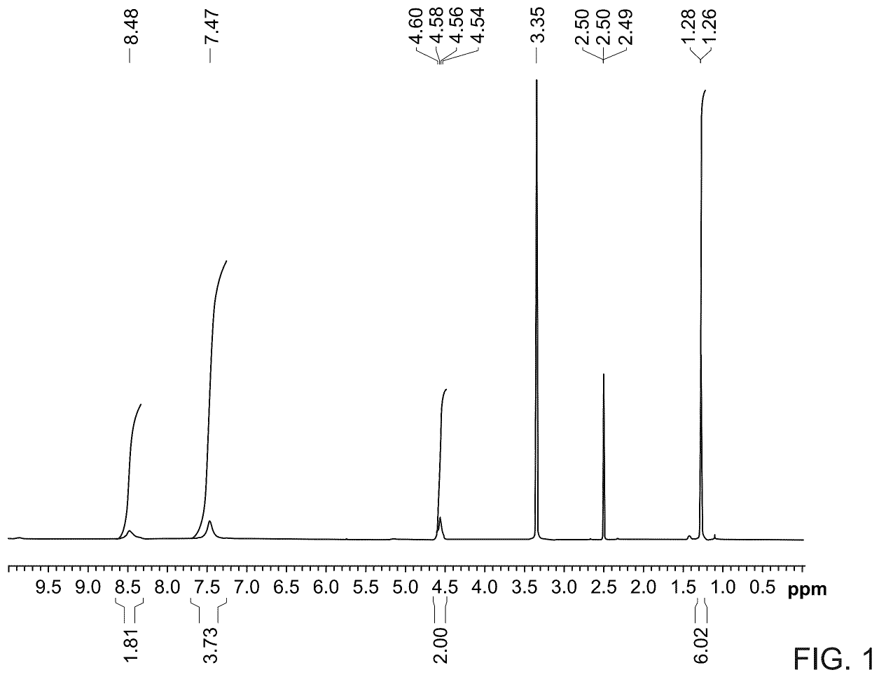

FIG. 2 depicts the 1H NMR spectrum of vorasidenib free form.

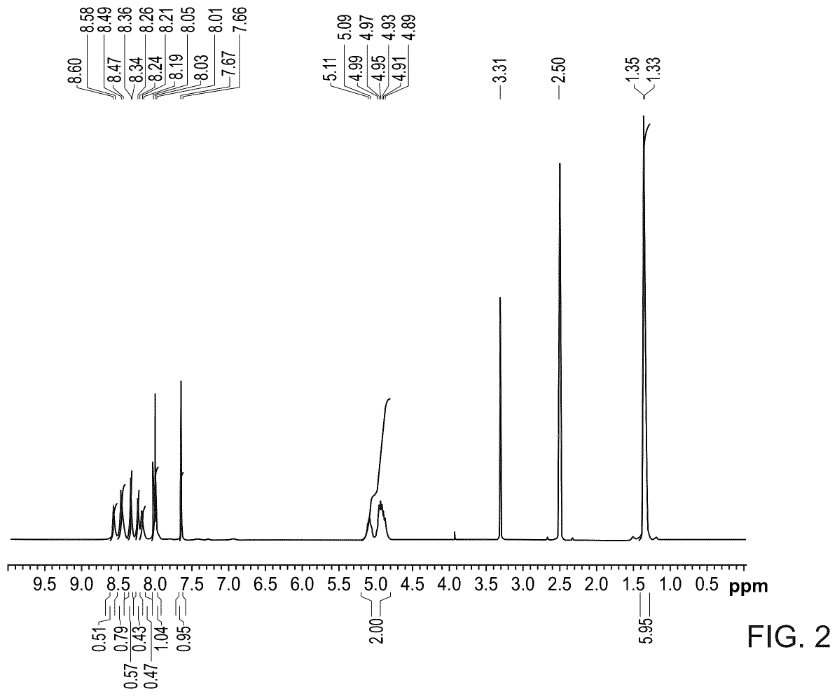

### DETAILED DESCRIPTION

The present disclosure relates to a method for preparation of vorasidenib of Formula (I).

A synthesis of vorasidenib of formula (I), that is 6-(6-chloropyridin-2-yl)-N²,N⁴-bis((R)-1,1,1-trifluoropropan-2-yl)-1,3,5-triazine-2,4-diamine, is described in PCT publication WO2015/003640 (Example 10). Vorasidenib is prepared according to the following reaction scheme:

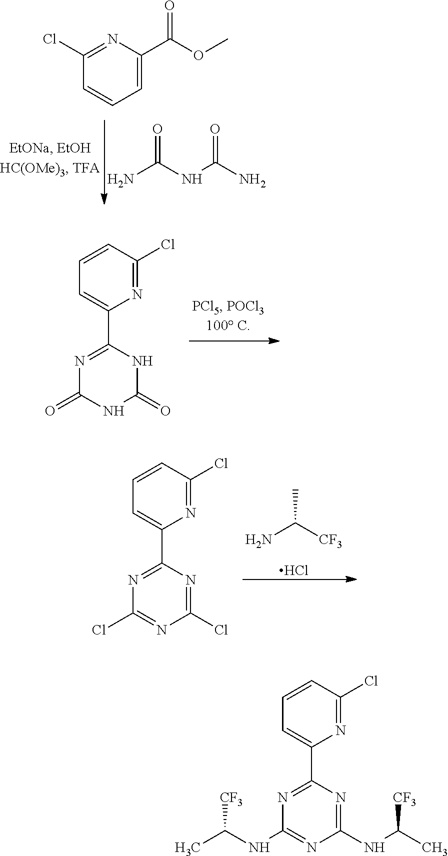

An alternative method for preparation of vorasidenib of formula (I) is disclosed in paragraphs [00312]-[00320](Example 10) of PCT publication WO2019/090059 according to the following reaction scheme:

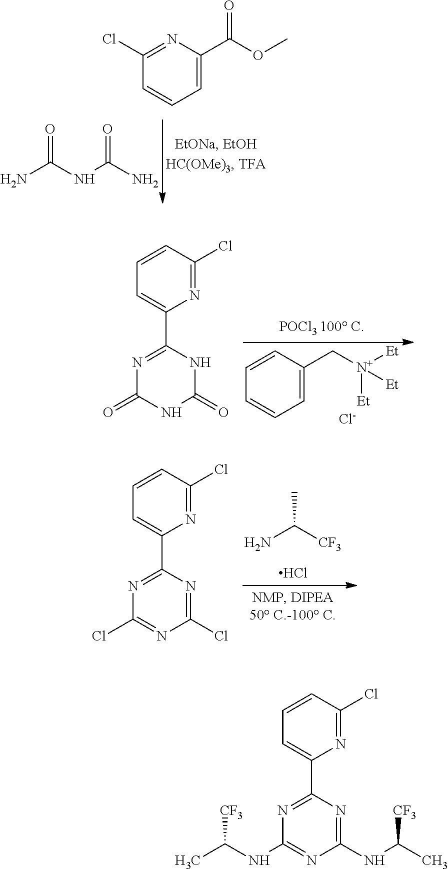

This alternative method for preparation of vorasidenib yields the target compound with a global yield of 30%.

Both methods of the prior art use 2,4-dichloro-6-(6-chloropyridin-2-yl)-1,3,5-triazine as intermediate compound. Said 2,4-dichloro-6-(6-chloropyridin-2-yl)-1,3,5-triazine is not very stable and should be stored in solution. In addition, use of POCl₃ as a reagent at industrial scale is difficult to manage, due to its high toxicity (it may be fatal by inhalation) and the formation of phosphoric acid and hydrochloric acid during its hydrolysis. Its industrial use is strictly regulated in the EU.

The present invention discloses a method for preparation of vorasidenib that may be used at an industrial scale with limited safety issues. The method of the present invention provides vorasidenib in a good yield and minimizes the number of reaction steps. The key intermediate of this alternative synthesis is N,N′-bis[(2R)-1,1,1-trifluoropropan-2-yl]triimidodicarbonic diamide hydrochloride of formula (III).

Some documents of the prior art disclose the preparation of guanidines, either symmetric or non-symmetric (for example: Grytsai, O. et al. Beilstein J. Org. Chem. 2021, 17, 1001-1040, LeBel, O. et al. Can. J. Chem. 2005, 83, 615-625, Nandini R. Pai and Seema S. Sawant. Der Pharma Chemica, 2014, 6, 176-187, WO 2016/175357 A1, IMMUNOMET THERAPEUTICS INC. and WO2015/003 640 A1).

The present invention relates to a process for the preparation of vorasidenib of formula (I):

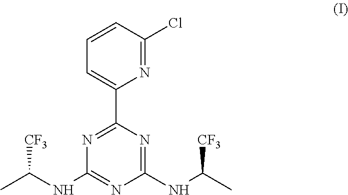

comprising reacting (R)-1,1,1-trifluoropropan-2-amine hydrochloride of formula (II):

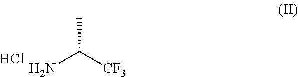

with sodium dicyanamide,
in either an alcoholic solvent selected from methanol, isopropanol, isobutanol, tert-butanol, tert-amyl alcohol (i.e. 2-methyl butan-2-ol), cyclopentanol and cyclohexanol, or in isobutyl acetate at a temperature between 65° C. and 140° C.,
to yield N,N′-bis[(2R)-1,1,1-trifluoropropan-2-yl]triimidodicarbonic diamide hydrochloride of formula (III)

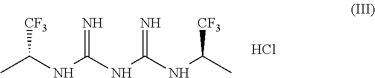 [SMILES: C[C@@H](NC(=N)NC(=N)N[C@H](C)C(F)(F)F)C(F)(F)F ; Cl]

followed by reacting the compound of formula (III) with methyl 6-chloropyridine-2-carboxylate, in a solvent selected from alcohols and acetonitrile at a temperature between 20° C. and 80° C., in the presence of a base,
to yield vorasidenib.

The temperature referred to in this application is the real temperature of the reaction medium.

In one embodiment, the reaction between the compound of formula (II) and sodium dicyanamide is performed at a temperature between 85° C. and 140° C.

In one embodiment, the solvent used to perform the reaction between the compound of formula (II) and sodium dicyanamide is an alcoholic solvent selected from tert-butanol, tert-amyl alcohol (i.e. 2-methyl butan-2-ol), isopropanol and cyclohexanol.

In one embodiment, the solvent used to perform the reaction between the compound of formula (II) and sodium dicyanamide is selected from tert-butanol and tert-amyl alcohol (i.e. 2-methyl butan-2-ol).

In one embodiment, the reaction between the compound of formula (II) and sodium dicyanamide is performed at a concentration between 8 mL/g and 34 mL/g calculated from the amount of sodium dicyanamide.

In one embodiment, the quantity of compound of formula (II) used in the reaction with sodium dicyanamide is between 1.9 and 3 equivalents calculated from the molar quantity of sodium dicyanamide.

In another embodiment, the quantity of compound of formula (II) used in the reaction with sodium dicyanamide is between 1.9 and 2.5 equivalents calculated from the molar quantity of sodium dicyanamide.

In one embodiment, the solvent used to perform the reaction between the compound of formula (III) and methyl 6-chloropyridine-2-carboxylate is an alcohol selected from methanol, ethanol, isopropanol, tert-butanol and a mixture of methanol and tert-butanol.

In one embodiment, the solvent used to perform the reaction between the compound of formula (III) and methyl 6-chloropyridine-2-carboxylate is acetonitrile.

In one embodiment, the base used to perform the reaction between the compound of formula (III) and methyl 6-chloropyridine-2-carboxylate is selected from MeONa, tBuOK (i.e., potassium tert-butoxide), K₂CO₃, Cs₂CO₃, NaOH, KOH, LiOH and K₃PO₄.

In a preferred embodiment, the solvent used to perform the reaction between the compound of formula (III) and methyl 6-chloropyridine-2-carboxylate is methanol and the base used to perform the reaction between the compound of formula (III) and methyl 6-chloropyridine-2-carboxylate is MeONa.

In one embodiment, the quantity of the base used to perform the reaction between the compound of formula (III) and methyl 6-chloropyridine-2-carboxylate is between 0.8 and 2 equivalents calculated from the molar quantity of compound of formula (III).

In a preferred embodiment, the quantity of the base used to perform the reaction between the compound of formula (III) and methyl 6-chloropyridine-2-carboxylate is between 1.5 and 2 equivalents calculated from the molar quantity of compound of formula (III).

In one embodiment, the reaction between the compound of formula (III) and methyl 6-chloropyridine-2-carboxylate is performed at a concentration between 5 mL/g and 10 mL/g calculated from the amount of compound of formula (III).

In one embodiment, the quantity of methyl 6-chloropyridine-2-carboxylate used in the reaction with compound of formula (III) is between 0.8 and 3 equivalents calculated from the molar quantity of compound of formula (III).

In a preferred embodiment, the quantity of methyl 6-chloropyridine-2-carboxylate used in the reaction with compound of formula (III) is between 1.5 and 2.5 equivalents calculated from the molar quantity of compound of formula (III).

In a further preferred embodiment, quantity of methyl 6-chloropyridine-2-carboxylate used in the reaction with compound of formula (III) is between 1.5 and 2.2 equivalents calculated from the molar quantity of compound of formula (III).

In one embodiment, the reaction between the compound of formula (III) and methyl 6-chloropyridine-2-carboxylate is performed at a temperature between 20° C. and 50° C., preferably at a temperature between 20° C. and 40° C.

Advantageously, both steps of the process can be performed sequentially without any isolation and purification of the compound of formula (III).

The present invention also relates to the use of N,N′-bis[(2R)-1,1,1-trifluoropropan-2-yl]triimidodicarbonic diamide hydrochloride as an intermediate compound for the preparation of vorasidenib.

### EMBODIMENTS

E1. A process for the preparation of vorasidenib of formula (I)

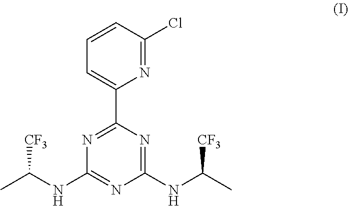

comprising reacting (R)-1,1,1-trifluoropropan-2-amine hydrochloride of formula (II):

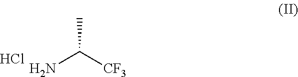

with sodium dicyanamide,
in either an alcoholic solvent selected from methanol, isopropanol, isobutanol, tert-butanol, tert-amyl alcohol (i.e. 2-methyl butan-2-ol), cyclopentanol and cyclohexanol, or in isobutyl acetate at a temperature between 65° C. and 140° C.,
to yield N,N′-bis[(2R)-1,1,1-trifluoropropan-2-yl]triimidodicarbonic diamide hydrochloride of formula (III)

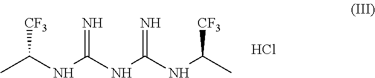 [SMILES: C[C@@H](NC(=N)NC(=N)N[C@H](C)C(F)(F)F)C(F)(F)F ; Cl]

followed by reacting the compound of formula (III) with methyl 6-chloropyridine-2-carboxylate, in a solvent selected from alcohols and acetonitrile at a temperature between 20° C. and 80° C., in the presence of a base,
to yield vorasidenib.

E2. Process according to E1, wherein the solvent used to perform the reaction between the compound of formula (II) and sodium dicyanamide is an alcoholic solvent selected from tert-butanol, tert-amyl alcohol (i.e. 2-methyl butan-2-ol), isopropanol and cyclohexanol.

E3. Process according to E2, wherein the solvent used to perform the reaction between the compound of formula (II) and sodium dicyanamide is tert-butanol or tert-amyl alcohol (i.e. 2-methyl butan-2-ol).

E4. Process according to any one of E1-E3, wherein the reaction between the compound of formula (II) and sodium dicyanamide is performed at a temperature between 85° C. and 140° C.

E5. Process according to any one of E1-E4, wherein the solvent used to perform the reaction between the compound of formula (III) and methyl 6-chloropyridine-2-carboxylate is an alcohol selected from methanol, ethanol, isopropanol, tert-butanol and a mixture of methanol and tert-butanol.

E6. Process according to any one of E1-E5, wherein the base used to perform the reaction between the compound of formula (III) and methyl 6-chloropyridine-2-carboxylate is selected from MeONa, tBuOK (i.e., potassium tert-butoxide), K₂CO₃, Cs₂CO₃, NaOH, KOH, LiOH and K₃PO₄.

E7. Process according to any one of E1-E6, wherein the solvent used to perform the reaction between the compound of formula (III) and methyl 6-chloropyridine-2-carboxylate is methanol and the base used to perform the reaction between the compound of formula (III) and methyl 6-chloropyridine-2-carboxylate is MeONa or tBuOK (i.e., potassium tert-butoxide).

E8. Process according to any one of E1-E6, wherein the solvent used to perform the reaction between the compound of formula (III) and methyl 6-chloropyridine-2-carboxylate is a mixture of methanol and tert-butanol and the base used to perform the reaction between the compound of formula (III) and methyl 6-chloropyridine-2-carboxylate is MeONa.

E9. Process according to any one of E1-E6, wherein the solvent used to perform the reaction between the compound of formula (III) and methyl 6-chloropyridine-2-carboxylate is ethanol and the base used to perform the reaction between the compound of formula (III) and methyl 6-chloropyridine-2-carboxylate is K₂CO₃ or KOH.

E10. Process according to any one of E1-E9, wherein the reaction between the compound of formula (III) and methyl 6-chloropyridine-2-carboxylate is performed at a temperature between 20° C. and 50° C., preferably at a temperature between 20° C. and 40° C.

E11. Process according to E1, wherein:
the solvent used to perform the reaction between the compound of formula (II) and sodium dicyanamide is tert-butanol or tert-amyl alcohol (i.e. 2-methyl butan-2-ol),
the reaction between the compound of formula (II) and sodium dicyanamide is performed at a temperature between 85° C. and 140° C.,
the solvent used to perform the reaction between the compound of formula (III) and methyl 6-chloropyridine-2-carboxylate is methanol,
the base used to perform the reaction between the compound of formula (III) and methyl 6-chloropyridine-2-carboxylate is MeONa,
the reaction between the compound of formula (III) and methyl 6-chloropyridine-2-carboxylate is performed at a temperature between 20° C. and 50° C.

E12. Process according to E11, characterized in that/wherein the mixture of compound of formula (II) and sodium dicyanamide is first heated at a temperature between 35° C. and 45° C. before being heated at a temperature between 85° C. and 140° C.

E13. Process according to any one of E1-E12, wherein the solvent used to perform the reaction between the compound of formula (II) and sodium dicyanamide is tert-amyl alcohol (i.e. 2-methyl butan-2-ol).

E14. Process according to any one of E1-E12, wherein the solvent used to perform the reaction between the compound of formula (II) and sodium dicyanamide is tert-butanol.

E15. Process according to any one of E1-E14, wherein the reaction between the compound of formula (II) and sodium dicyanamide is performed at a concentration between 8 mL/g and 34 mL/g calculated from the amount of sodium dicyanamide.

E16. Process according to any one of E1-E15, wherein the quantity of compound of formula (II) used in the reaction with sodium dicyanamide is between 1.9 and 3 equivalents calculated from the molar quantity of sodium dicyanamide.

E17. Process according to E16, wherein the quantity of compound of formula (II) used in the reaction with sodium dicyanamide is between 1.9 and 2.5 equivalents calculated from the molar quantity of sodium dicyanamide.

E18. Process according to any one of E1-E17, wherein the quantity of the base used to perform the reaction between the compound of formula (III) and methyl 6-chloropyridine-2-carboxylate is between 0.8 and 2 equivalents calculated from the molar quantity of compound of formula (III).

E19. Process according to E18, wherein the quantity of the base used to perform the reaction between the compound of formula (III) and methyl 6-chloropyridine-2-carboxylate is between 1.5 and 2 equivalents calculated from the molar quantity of compound of formula (III).

E20. Process according to any one of E1-E19, wherein the reaction between the compound of formula (III) and methyl 6-chloropyridine-2-carboxylate is performed at a concentration between 5 mL/g and 10 mL/g calculated from the amount of compound of formula (III).

E21. Process according to any one of E1-E20, wherein the quantity of methyl 6-chloropyridine-2-carboxylate used in the reaction with compound of formula (III) is between 0.8 and 3 equivalents calculated from the molar quantity of compound of formula (III).

E22. Process according to E21, wherein the quantity of methyl 6-chloropyridine-2-carboxylate used in the reaction with compound of formula (III) is between 1.5 and 2.5 equivalents calculated from the molar quantity of compound of formula (III).

E23. Process according to E22, wherein the quantity of methyl 6-chloropyridine-2-carboxylate used in the reaction with compound of formula (III) is between 1.5 and 2.2 equivalents calculated from the molar quantity of compound of formula (III).

E24. Use of N,N′-bis[(2R)-1,1,1-trifluoropropan-2-yl]triimidodicarbonic diamide hydrochloride of formula (III) for the preparation of vorasidenib.

E25. Process according to E1, wherein the compound of formula (III) obtained from the reaction of (R)-1,1,1-trifluoropropan-2-amine hydrochloride of formula (II) with sodium dicyanamide is not subjected to any purification or isolation before reacting with methyl 6-chloropyridine-2-carboxylate.

E26. Process according to E25, wherein the solvent used to perform the reaction between the compound of formula (II) and sodium dicyanamide is tert-butanol and the solvent used to perform the reaction between the compound of formula (III) and methyl 6-chloropyridine-2-carboxylate is a mixture of methanol and tert-butanol.

E27. Process according to E25 or E26, wherein the reaction between the compound of formula (II) and sodium dicyanamide is:
performed at a temperature between 85° C. and 140° C.,
performed at a concentration between 8 mL/g and 34 mL/g calculated from the amount of sodium dicyanamide.

E28. Process according to any one of E25-E27, wherein the quantity of compound of formula (II) used in the reaction with sodium dicyanamide is between 1.9 and 3 equivalents calculated from the molar quantity of sodium dicyanamide, preferably between 1.9 and 2.5 equivalents calculated from the molar quantity of sodium dicyanamide.

E29. Process according to any one of E25-E28, wherein the base used to perform the reaction between the compound of formula (III) and methyl 6-chloropyridine-2-carboxylate is MeONa, used in a quantity between 0.8 and 2 equivalents calculated from the molar quantity of sodium dicyanamide, preferably between 1.5 and 2 equivalents calculated from the molar quantity of sodium dicyanamide.

E30. Process according to any one of E25-E29, wherein the quantity of methyl 6-chloropyridine-2-carboxylate used in the reaction with the compound of formula (III) is between 0.8 and 3 equivalents calculated from the molar quantity of sodium dicyanamide, preferably between 1.5 and 2.5 equivalents calculated from the molar quantity of sodium dicyanamide, preferably between 1.5 and 2.2 equivalents calculated from the molar quantity of sodium dicyanamide.

E31. Process according to any one of E25-E30, wherein the reaction between the compound of formula (III) and methyl 6-chloropyridine-2-carboxylate is performed at a concentration between 5 mL/g and 10 mL/g calculated from the theoretical amount of compound of formula (III) that is obtained when the reaction between the compound of formula (II) and sodium dicyanamide is complete.

E32. Process according to any one of E25-E31, wherein the reaction between the compound of formula (III) and methyl 6-chloropyridine-2-carboxylate is performed at a temperature between 20° C. and 50° C., preferably at a temperature between 20° C. and 40° C.

E33. Process according to any one of E1-E32, wherein the vorasidenib of formula (I) is further transformed into a hemicitric acid hemihydrate cocrystal.

### EXAMPLES

### Abbreviations

| | |
|---|---|
| aq. | aqueous |
| DIPEA | N,N-diisopropylethylamine |
| DMSO | dimethylsulfoxide |
| eq. | equivalent |
| EtOH | Ethanol |
| IPC | In Progress Control |
| iPrOH | Isopropyl alcohol (i.e., propan-2-ol) |
| LC | Liquid chromatography |
| MeCN | acetonitrile |
| MeOH | Methanol |
| MeONa | Sodium methylate (i.e., sodium methoxide) |
| MSA | methanesulfonic acid |
| N/A | Not applicable |
| n.d. | Not determined |
| NMP | N-methylpyrrolidone |
| NMR | Nuclear Magnetic Resonance |
| tBuOK | Potassium tert-butylate (i.e., Potassium tert-butoxide) |
| T | Temperature |
| s | singlet |
| d | doublet |
| t | triplet |
| m | multiplet |
| br | broad |

¹H Liquid NMR spectra were collected on a Bruker 400 MHz NMR Spectrometer. The chemical shifts, in ppm, are given with respect to tetramethylsilane (TMS), using partially deuterated dimethylsulfoxide as internal standard. In solution in partially deuterated dimethylsulfoxide, the resonance at 2.5 ppm on the 1D 1H NMR spectrum is due to partially deuterated dimethylsulfoxide and the resonance at 3.30 ppm is due to the presence of water.

In this experimental section, the following terms will be used:
(R)-1,1,1-trifluoropropan-2-amine hydrochloride of formula (II) will be named Cpd 2,
N,N′-bis[(2R)-1,1,1-trifluoropropan-2-yl]triimidodicarbonic diamide hydrochloride of formula (III) will be named Cpd 3,
methyl 6-chloropyridine-2-carboxylate will be named Cpd 4.

### LC Conditions

Column: Acquity UPLC BEH C18 (50 mm×2.1 mm)-injected volume: 0.5 μL-Analysis time: 5.6 min-temperature: 40° C.-wavelength: 210 nm-eluent: phase A H₂O/MeCN/MSA (1000:25:1), phase B H₂O/MeCN/MSA (25:1000:1)-elution: t(0-1 min) A 95%, B 5%; t(1-4.5 min) A 95%, B 5%→A 5%, B 95%; t(4.5-5.5 min) A 5%, B 95%-flow: 0.8 mL/min.

### Retention Times

Cpd2: 1.50 min
Cpd 3: 2.03 min
Vorasidenib free form: 2.85 min

### Example 1: Screening of conditions to prepare N,N′-bis[(2R)-1,1,1-trifluoropropan-2-yl]triimidodicarbonic diamide hydrochloride

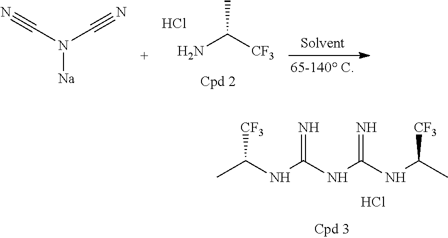

A reactor equipped with a mechanical stirrer under a nitrogen atmosphere was charged with sodium dicyanamide (1.8 g, 20.1 mmol, 1.0 eq), Cpd 2 (2.1 eq to 3 eq, refer to Table 1) and solvent (8 to 34 mL/g calculated from the amount of sodium dicyanamide) at 35° C. The reaction mixture was heated to 65-115° C. (refer to Table 1) and the reaction mixture was stirred at this temperature for the time indicated in Table 1 below. After cooling to 35° C., the reaction progress was monitored by LC.

TABLE 1

| Solvent | T | Cpd | Time | IPC by LC | Yield | Purity by LC |
|---|---|---|---|---|---|---|
| (Dilution (mL/g)) | (° C.) | 2 eq. | (h) | % Cpd 3 | (%) | % Cpd 3 |
| Isobutyl acetate* | 115 | 2.1 | 18 | 63.9 | n.d. | N/A |
| (34) | | | | | | |
| MeOH | 115 | 2.1 | 18 | 70.2 | n.d. | N/A |
| (17) | | | | | | |
| iPrOH | 65 | 2 | 96 | 58.7 | n.d. | N/A |
| (34) | | | | | | |
| iPrOH | 75 | 3 | 36 | 45.6 | 85% | 85.0 |
| (17) | | | | | | |
| iPrOH | 115 | 2.1 | 18 | 69.8 | n.d. | N/A |
| (17) | | | | | | |
| n-butanol | 105 | 2.5 | 18 | 26.6 | n.d. | N/A |
| (17) | | | | | | |
| Isobutanol | 115 | 2.1 | 18 | 67.0 | n.d. | N/A |
| (17) | | | | | | |
| tert-butanol | 115 | 2.1 | 3 | 75.0 | 76.8 | 98.1 |
| (17) | | | | | | |
| tert-butanol | 110 | 3 | 4 | n.d | 76.0 | 85.3 |
| (34) | | | | | | |
| tert-butanol | 115 | 2.3 | 8 | 82.0 | 71.1 | 93 |
| (8) | | | | | | |
| tert-amyl alcohol | 115 | 2.5 | 3 | 79.8 | 76 | 98.2 |
| (17) | | | | | | |
| tert-amyl alcohol | 100 | 2.3 | 8 | 82.1 | 64.8 | 95.5 |
| (10) | | | | | | |
| Cyclopentanol | 115 | 2.1 | 18 | 70.2 | n.d. | N/A |
| (17) | | | | | | |
| Cyclohexanol | 115 | 2.5 | 3 | 66.9 | 68.4 | 97.6 |
| (17) | | | | | | |

*This experiment was performed on a 60 mg scale

### Example 2: Screening of conditions to prepare N,N′-bis[(2R)-1,1,1-trifluoropropan-2-yl]triimidodicarbonic diamide hydrochloride (Cpd 3) in tert-butanol

The same general protocol as the one used in Example 1 was used. The dilution of the reaction medium was set at 17 mL/g calculated from the amount of sodium dicyanamide.

TABLE 2

| | | | IPC by LC | | Purity by LC |
|---|---|---|---|---|---|
| T (° C.) | Cpd 2 eq. | Time (h) | % Cpd 3 | Yield (%) | % Cpd 3 |
| 85 | 2.5 | 12 | 84.2 | n.d. | N/A |
| 95 | 2.5 | 3 | 71.0 | 74.4 | 99.1 |
| 105 | 2.5 | 3 | 76.9 | 77.1 | 99.3 |
| 115 | 2.1 | 3 | 75.0 | 76.8 | 98.1 |
| 115 | 2.2 | 3 | 76.6 | 74.4 | 97.9 |
| 115 | 2.0 | 3 | 74.4 | 76.1 | 99.0 |
| 115 | 1.9 | 3 | 70.8 | 70.3 | 96.7 |
| 140 | 2.1 | 3 | 73.3 | 78.6 | 100 |

### Example 3: Screening of conditions to prepare vorasidenib free form from N,N′-bis[(2R)-1,1,1-trifluoropropan-2-yl]triimidodicarbonic diamide hydrochloride

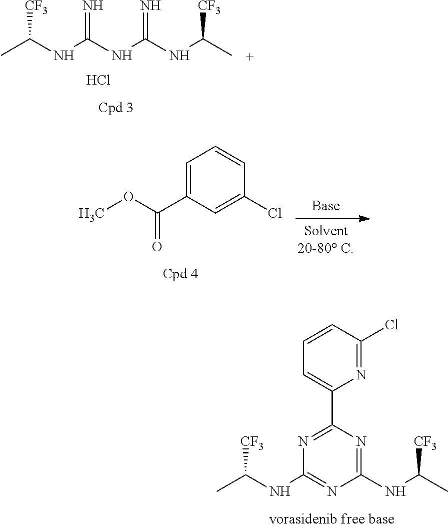 [SMILES: C[C@@H](NC(=N)NC(=N)N[C@H](C)C(F)(F)F)C(F)(F)F ; Cl ; COC(=O)c1cccc(Cl)c1 ; C[C@@H](Nc1nc(N[C@H](C)C(F)(F)F)nc(-c2cccc(Cl)n2)n1)C(F)(F)F]

A reactor equipped with a mechanical stirrer under a nitrogen atmosphere was charged with Cpd 3 (4 g, 12.1 mmol, 1.0 eq.), Cpd 4 (0.8 eq.-2 eq., refer to Table 3) and solvent (5 to 10 mL/g calculated from the amount of Cpd 3) at 20° C. The reaction mixture was heated to 20-80° C. (refer to Table 3) and base (1 eq.-2eq., refer to Table 3) was added dropwise (or portion wise for solid bases) to the reaction mixture. After stirring during the time disclosed in Table 3 at the said temperature, the reaction progress was monitored by LC.

Sometimes the reaction mixture was cooled to 20° C. and diluted with water (5 mL/g) and the mixture was stirred for 3 hours. The suspension was filtered, and the cake was washed with a mixture 1:1 of methanol (1 mL/g) and water (1 mL/g). Then the solid was dried at 40° C. in ventilated oven for 24 hours to provide vorasidenib free form.

TABLE 3

| | | | | | IPC by LC | | Purity by LC |
|---|---|---|---|---|---|---|---|
| Solvent | | | | | % of | | % of |
| (Dilution | Cpd 4 | Base | T | Time | vorasidenib | Yield | vorasidenib |
| (mL/g)) | (eq.) | (eq.) | (° C.) | (h) | free form | (%) | free form |
| MeOH | 2 | MeONa | 20 | 18 | 82.8 | 75.9 | 100 |
| (8) | | (2) | | | | | |
| MeOH | 0.8 | MeONa | 20 | 18 | 77.1 | 38 | 100 |
| (8) | | (2) | | | | | |
| MeOH | 1 | MeONa | 20 | 18 | 78.1 | 53 | 100 |
| (8) | | (2) | | | | | |
| MeOH | 1.2 | MeONa | 20 | 18 | 82.7 | 62.5 | 99.2 |
| (8) | | (2) | | | | | |
| MeOH | 1.5 | MeONa | 20 | 1 | 83.3 | 62.2 | 99.4 |
| (8) | | (2) | | | | | |
| MeOH | 1.5 | MeONa | 40 | 2 | 77.4 | n.d. | N/A |
| (5) | | (1) | | | | | |
| MeOH | 1.5 | K₂CO₃ | 40 | 1 | 51.3 | n.d. | N/A |
| (5) | | (1.1) | | | | | |
| MeOH | 1.5 | Cs₂CO₃ | 40 | 1 | 49.7 | n.d. | N/A |
| (5) | | (1.1) | | | | | |
| MeOH | 1.5 | K₃PO₄ | 40 | 1 | 55.9 | n.d. | N/A |
| (5) | | (1.1) | | | | | |
| MeOH | 1.5 | LiOH | 40 | 1 | 54.4 | n.d. | N/A |
| (5) | | (1.1) | | | | | |
| MeOH | 1.5 | tBuOK | 40 | 1 | 74.6 | n.d. | N/A |
| (5) | | (1.1) | | | | | |
| MeOH | 1.5 | tBuOK | 30 | 2.5 | 43.7 | n.d. | N/A |
| (8) | | (2) | | | | | |
| EtOH | 1.5 | K₂CO₃ | 40 | 2 | 75.3 | n.d. | N/A |
| (5) | | (1.5) | | | | | |
| EtOH | 1.5 | KOH | 40 | 2 | 68.7 | n.d | N/A |
| (5) | | (1.5) | | | | | |
| EtOH | 1.5 | NaOH | 40 | 2 | 25.6 | n.d. | N/A |
| (5) | | aq. (1.5) | | | | | |
| tert-butanol | 1.5 | tBuOK | 30 | 2.5 | 44.5 | n.d | N/A |
| (8) | | (2) | | | | | |
| Isopropanol | 1.5 | tBuOK | 30 | 2.5 | 16.3 | n.d. | N/A |
| (8) | | (2) | | | | | |
| Isopropanol | 1.5 | MeONa | 80 | 18 | 87.2 | n.d. | N/A |
| (5) | | (1.5) | | | | | |
| MeCN | 1.5 | K₂CO₃ | 40 | 18 | 41 | n.d. | N/A |
| (5) | | (1.5) | | | | | |

### Example 4: Synthesis of vorasidenib hemicitric acid hemihydrate Cocrystal

### Step 1: Preparation of N,N′-bis[(2R)-1,1,1-trifluoropropan-2-yl]triimidodicarbonic diamide hydrochloride of formula (III)

An 8 L reactor equipped with a mechanical stirrer under a nitrogen atmosphere was charged with sodium dicyanamide (0.35 kg, 3.9 mol, 1.0 eq.), Cpd 2 (1.25 kg, 8.3 mol, 2.1 eq.) and tert-butanol (5.9 L, 16.7 vol) at 35° C. The reaction mixture was heated to 130° C. and the reaction mixture was stirred at 130° C. for 4 hours. After cooling to 35° C., the reaction progress was monitored by LC.

At 35° C., the reaction mixture was diluted with isopropanol (2.4 L, 7 vol) and the mixture was stirred for 30 minutes. The suspension was filtered, and the cake was washed with isopropanol (0.7 L, 2 vol). Then the solid was dried at 40° C. in ventilated oven for 72 hours to provide Cpd 3 (1.03 kg, 3.1 mol, yield 80%, purity LC 83%)

1H NMR analysis of Cpd 3

¹H NMR (400 MHz; DMSO-d6) δ: 8.48 (br s, 2H), 7.47 (br s, 4H), 4.57 (m, 2H), 1.27 (d, J=8.0 Hz, 6H).

### Step 2: Preparation of Vorasidenib

A 22 L reactor equipped with a mechanical stirrer under a nitrogen atmosphere was charged with Cpd 3 (1 kg, 3.0 mol, 1.0 eq.), Cpd 4 (0.78 kg, 4.5 mol, 1.5 eq.) and methanol (5 L, 5 vol) at 20° C. The reaction mixture was heated to 50° C. and sodium methylate (i.e., sodium methoxide) (30% in methanol, 1.1 kg, 6.0 mol, 2.0 eq.) was added dropwise to the reaction mixture. After stirring 3 h at 50° C. the reaction progress was monitored by LC. The reaction mixture was cooled to 20° C. and diluted with water (5 L, 5 vol) and the mixture was stirred for 3 hours. The suspension was filtered, and the cake was washed with a mixture 1:1 of methanol (1.5 L, 1.5 V) and water (1.5 L, 1.5 vol). Then the solid was dried at 40° C. in ventilated oven for 24 hours to provide vorasidenib free form (0.79 kg, 1.9 mol, yield 69%, purity LC 99%).

¹H NMR analysis of vorasidenib

¹H NMR (400 MHz; DMSO-d6) δ: 8.59, 8.48 and 8.20 (m, 2H), 8.35 and 8.25 (m, 1H), 8.03 (t, J=8 Hz, 1H), 7.67 (d, J=4 Hz, 1H), 5.11-4.89 (m, 2H), 1.34 (d, J=8.0 Hz, 6H).

### Step 3: Preparation of Cocrystal of Vorasidenib with Citric Acid

A 500 mL reactor equipped with a mechanical stirrer under a nitrogen atmosphere was charged with a solution of acetone/n-heptane 9:1 v/v (0.26 kg), and vorasidenib free form obtained according to procedures disclosed in steps 1 and 2 (57.6 g, 0.14 mol, 1.0 eq.). Once dissolution was complete, citric acid monohydrate (15 g, 0.07 mol, 0.51 eq.) was added at 30° C. to the reaction mixture which was stirred until complete dissolution. The reaction mixture was polish filtered and transfer to another reactor. A rinse was performed with a solution of acetone/n-heptane 9:1 v/v (32 g).

n-heptane (51.6 g) was added to the polished solution over 1 h. Then, vorasidenib hemicitric acid hemihydrate cocrystal seeds (1.26 g, 1.21 mmol, 0.8% eq.) were added around 30° C. and the mixture was stirred 45 min at 30° C. Then, n-heptane (0.62 kg) was added over 4.5 h to control the formation of the co-crystal. The suspension was maintained at 32° C. for 1 h, cooled to 10° C. over 2.5 h and stirred at 10° C. for 12 hours. Then the suspension was wet milled to reach PSD specifications. Finally, the slurry was filtered, and the cake was washed two times with a mixture of acetone/n-heptane 1:3.2 v/v (98 g). The wet solid was dried under vacuum around 30° C. for 4 days to afford vorasidenib hemicitric acid hemihydrate cocrystal (64.7 g, 111 mol, yield 90%, purity LC 99%).

### Example 5: One-Pot Preparation of Vorasidenib Free Form

### A. Reaction Conditions for Step 1, Example 1

A 100 mL reactor equipped with a mechanical stirrer under a nitrogen atmosphere was charged with sodium dicyanamide (1.8 g, 20.1 mmol, 1.0 eq.), Cpd 2 (7.5 g, 50.2 mmol, 2.1 eq.) and tert-butanol (30 mL, 16.7 vol) at 25° C. The reaction mixture was heated to 115° C. and the reaction mixture was stirred at 115° C. for 3 hours. After cooling to 30° C. the reaction progress was monitored by LC until the quantity of Cpd 2 was ≤5%. The reaction mixture was half concentrated and engaged to second step without any isolation and purification.

### B. Reaction Conditions for Step 2, Example 1

A 250 mL reactor equipped with a mechanical stirrer under a nitrogen atmosphere was charged with a solution of Cpd 3 in tert-butanol (total volume 20 mL), Cpd 4 (5.2 g, 30.1 mmol, 1.5 eq.) and methanol (30 mL, 16.7 vol) at 20° C. The reaction mixture was heated to 40° C. and sodium methylate (i.e., sodium methoxide) (25% in methanol, 8.6 mL, 37.6 mmol, 1.9 eq.) was added over 2 hours to the reaction mixture. After stirring 2 h at 40° C. the reaction progress was monitored by LC until the quantity of Cpd 3 was ≤5%.

The equivalents in the second steps are calculated from the molar quantity of sodium dicyanamide used in step A, considering that the full conversion of the said sodium dicyanamide in Cpd 3 occurs during step A.

The concentration of the reaction medium during step B is between 5 mL/g and 10 mL/g calculated from the theoretical amount of Cpd 3 that would be obtained if step A had a 100% yield (reaction complete).

### C. Work-Up/Isolation

The reaction mixture was cooled to 20° C. and diluted with water (59.5 mL, 33.3 vol) and the mixture was stirred for 12 hours. The suspension was filtered, and the cake was washed with a mixture 1:4.2 of methanol (5.4 mL, 3 vol) and water (17.9 L, 10 vol). Then the solid was dried at 50° C. in ventilated oven for 24 hours to provide vorasidenib free form (4.29 g, 10.3 mmol, yield 52%, purity LC 99%).

### Example 6: Preparation of N,N′-bis[(2R)-1,1,1-trifluoropropan-2-yl]triimidodicarbonic diamide hydrochloride of formula (III) according to the disclosure of Nandini R. Pai et al. (Der Pharma Chemica, 2014, 6, 176-187)

Sodium dicyanamide (0.3 g, 3.3 mmol, 1.0 eq.), Cpd 2 (2 eq.) are brought together either in n-butanol or in isobutanol to reproduce the reaction disclosed in the publication by Nandini R. Pai et al.

The results are summarized in the table below

| | | | | | | Purity (LC) |
|---|---|---|---|---|---|---|
| | T | Cpd 2 | Time | IPC by LC | Yield | % of isolated |
| Solvent | (° C.) | (eq.) | (h) | % of Cpd 3 | (%) | Cpd 3 |
| n-butanol* | 80 | 2 | 20 | 46.3 | 32% | 93% |
| isobutanol | 80 | 2 | 36 | 23.5 | n.d. | N/A |

*A few drops of HCl need to be added in the mixture.

Despite a prolonged time of contact, the reaction occurs only partially in both cases not allowing for isolation of Cpd 3 in good yield.

### Example 7: Preparation N,N′-bis[(2R)-1,1,1-trifluoropropan-2-yl]triimidodicarbonic diamide hydrochloride according to the process disclosed in WO 2015/003640

Sodium dicyanamide (2 g, 22.5 mmol, 1.0 eq) and Cpd 2 (6.7 g, 44.9 mmol, 2 eq.) were heated at 160° C. for 18 h. Then, the reaction mixture was cooled to 25° C. and diluted with water (5 mL/g) and isopropanol (5 mL/g). Only 4% of Cpd 3 was observed in the mixture when performing the analysis by LC.

These operating conditions are not efficient to prepare Cpd 3.

### Example 8: Optimized conditions for preparation of N,N′-bis[(2R)-1,1,1-trifluoropropan-2-yl]triimidodicarbonic diamide hydrochloride according to the process disclosed in WO 2015/003640

To improve the process disclosed in Example 7, optimized conditions have been tested, especially through an adjusted quantity of Cpd 2 (2.35 eq) and lower temperature (around 95° C.).

Sodium dicyanamide (2 g, 22.5 mmol, 1.0 eq) and Cpd 2 (7.9 g, 52.8 mmol, 2.35 eq.) were heated at 40° C. for 12 h and at 95° C. (reflux) for 10 h. Then, the reaction mixture was cooled to 25° C. and diluted with water (10 mL/g) and isopropanol (10 mL/g). The suspension was filtered to afford only 4% of Cpd 3.

Even when parameters are optimized, these operating conditions, without solvent, do not allow for the preparation of Cpd 3 with good yield.

In addition, these conditions are not suitable for a scale-up and use at an industrial scale, due to the risk generated by heating powders at a high temperature.

### Example 9: Preparation of Vorasidenib at a Pilot Plant Scale

### Step 1: Preparation of N,N′-bis[(2R)-1,1,1-trifluoropropan-2-yl]triimidodicarbonic diamide hydrochloride of formula (III)

A 100 L reactor equipped with a mechanical stirrer under a nitrogen atmosphere was charged with sodium dicyanamide (2.50 kg, 28 mol, 1.0 eq), Cpd 2 (9.87 kg, 66 mol, 2.35 eq) and tert-amyl alcohol (25 L, 10 vol) at 25° C. The reaction mixture was stirred at 40° C. then 95° C. (reflux) until Cpd 2≤2% by LC.

### Work-Up/Isolation

At 25° C., the reaction mixture was diluted with water (18.75 L, 7.5 vol) and the mixture was stirred for 1 h. The suspension was filtered, and the cake was washed with isopropanol (12.5 L, 5 vol). Then the solid was dried at 50° C. in a ventilated oven for 48 hours to provide Cpd 3 (7.07 kg, 21 mol, yield 76.4%, purity LC >99%).

1H NMR analysis of Cpd 3

¹H NMR (400 MHz; DMSO-d6) δ: 8.48 (2H, NH), 7.47 (4H, NH), 4.57 (m, 2H, CH), 1.27 (d, J=8.0 Hz, 6H, CH₃).

### Step 2: Preparation of Vorasidenib

A 100 L reactor equipped with a mechanical stirrer under a nitrogen atmosphere was charged with Cpd 3 (7 kg, 21 mol, 1.0 eq), Cpd 4 (8.01 kg, 47 mol, 2.2 eq) and methanol (35 L, 5 vol) at 25° C. Sodium methylate (i.e., sodium methoxide) (30% in methanol, 7.64 kg, 42 mol, 2 eq.) was added dropwise to the reaction mixture over 1 h. After stirring 12 h at 25° C. the reaction progress was monitored by LC until Cpd 3≤1%.

### Work-Up/Isolation

The reaction mixture was diluted with water (35 L, 5 vol) and the mixture was stirred for 3 hours. The suspension was filtered, and the cake was washed with a mixture 1:1 of methanol (10.5 L, 1.5 V) and water (10.5 L, 1.5 vol). Then the solid was dried at 50° C. in the filter for 24 hours to provide vorasidenib free form (8.06 kg, 19 mol, yield 92%, purity LC >99%).

1H NMR analysis of vorasidenib

¹H NMR (400 MHz; DMSO-d6) δ: 8.59, 8.48 and 8.20 (2H, NH), 8.35 and 8.25 (1H, CH_{Ar}), 8.03 (t, J=8 Hz, 1H, CH_{Ar}), 7.67 (d, J=4 Hz, 1H, CH_{Ar}), 5.11-4.89 (m, 2H, CH), 1.34 (d, J=8.0 Hz, 6H, CH₃).

### Step 3: Preparation of Cocrystal of Vorasidenib with Citric Acid

A 500 mL reactor equipped with a mechanical stirrer under a nitrogen atmosphere was charged with a solution of acetone/n-heptane 9:1 v/v (0.26 kg), and vorasidenib free form (57.6 g, 0.14 mol, 1.0 eq). Once dissolution was complete, citric acid monohydrate (15 g, 0.07 mol, 0.51 eq) was added at 30° C. to the reaction mixture which was stirred until complete dissolution. The reaction mixture was polish filtered and transferred to another reactor. A rinse was performed with a solution of acetone/n-heptane 9:1 v/v (32 g). N-heptane (51.6 g) was added to the polished solution over 1 h. Then, vorasidenib hemicitric acid hemihydrate seeds (1.26 g, 1.21 mmol, 0.8% eq) were added around 30° C. and the mixture was stirred 45 min at 30° C. Then, n-heptane (0.62 kg) was added over 4.5 h to control the formation of the co-crystal. The suspension was maintained at 32° C. for 1 h, cooled to 10° C. over 2.5 h and stirred at 10° C. for 12 hours. Then the suspension was wet-milled. Finally, the slurry was filtered and the cake was washed two times with a mixture of acetone/n-heptane 1:3.2 v/v (98 g). The wet solid was dried under vacuum around 30° C. for 4 days to afford vorasidenib hemicitric acid hemihydrate (64.7 g, 111 mol, yield 89%, purity LC 99%).

1. A process for the preparation of vorasidenib of formula (I): 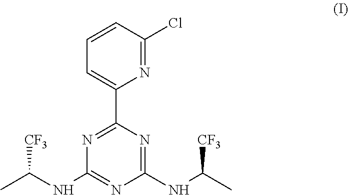
comprising reacting (R)-1,1,1-trifluoropropan-2-amine hydrochloride of formula (II): 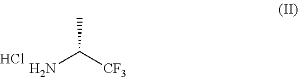
with sodium dicyanamide,
in either an alcoholic solvent selected from methanol, isopropanol, isobutanol, tert-butanol, tert-amyl alcohol (i.e. 2-methyl butan-2-ol), cyclopentanol and cyclohexanol, or in isobutyl acetate at a temperature between 65° C. and 140° C.,
to yield N,N′-bis[(2R)-1,1,1-trifluoropropan-2-yl]triimidodicarbonic diamide hydrochloride of formula (III) 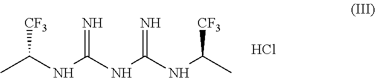 [SMILES: C[C@@H](NC(=N)NC(=N)N[C@H](C)C(F)(F)F)C(F)(F)F ; Cl]
followed by reacting the compound of formula (III) with methyl 6-chloropyridine-2-carboxylate,
in a solvent selected from alcohols and acetonitrile at a temperature between 20° C. and 80° C.,
in the presence of a base,
to yield vorasidenib.

2. The process according to claim 1, wherein the solvent used to perform the reaction between the compound of formula (II) and sodium dicyanamide is an alcoholic solvent selected from tert-butanol, tert-amyl alcohol (i.e. 2-methyl butan-2-ol), isopropanol and cyclohexanol.

3. The process according to claim 2, wherein the solvent used to perform the reaction between the compound of formula (II) and sodium dicyanamide is tert-butanol or tert-amyl alcohol (i.e. 2-methyl butan-2-ol).

4. The process according to claim 1, wherein the reaction between the compound of formula (II) and sodium dicyanamide is performed at a temperature between 85° C. and 140° C.

5. The process according to claim 1, wherein the solvent used to perform the reaction between the compound of formula (III) and methyl 6-chloropyridine-2-carboxylate is an alcohol selected from methanol, ethanol, isopropanol, tert-butanol and a mixture of methanol and tert-butanol.

6. The process according to claim 1, wherein the base used to perform the reaction between the compound of formula (III) and methyl 6-chloropyridine-2-carboxylate is selected from MeONa, tBuOK, K₂CO₃, Cs₂CO₃, NaOH, KOH, LiOH and K₃PO₄.

7. The process according to claim 1, wherein the solvent used to perform the reaction between the compound of formula (III) and methyl 6-chloropyridine-2-carboxylate is methanol and the base used to perform the reaction between the compound of formula (III) and methyl 6-chloropyridine-2-carboxylate is MeONa or tBuOK (i.e., potassium tert-butoxide).

8. The process according to claim 1, wherein the reaction between the compound of formula (III) and methyl 6-chloropyridine-2-carboxylate is performed at a temperature between 20° C. and 50° C.

9. The process according to claim 8, wherein the reaction between the compound of formula (III) and methyl 6-chloropyridine-2-carboxylate is performed at a temperature between 20° C. and 40° C.

10. The process according to claim 1, wherein:
the solvent used to perform the reaction between the compound of formula (II) and sodium dicyanamide is tert-butanol or tert-amyl alcohol (i.e. 2-methyl butan-2-ol),
the reaction between the compound of formula (II) and sodium dicyanamide is performed at a temperature between 85° C. and 140° C.,
the solvent used to perform the reaction between the compound of formula (III) and methyl 6-chloropyridine-2-carboxylate is methanol,
the base used to perform the reaction between the compound of formula (III) and methyl 6-chloropyridine-2-carboxylate is MeONa,
the reaction between the compound of formula (III) and methyl 6-chloropyridine-2-carboxylate is performed at a temperature between 20° C. and 50° C.

11. The process according to claim 1, wherein the solvent used to perform the reaction between the compound of formula (II) and sodium dicyanamide is tert-amyl alcohol (i.e. 2-methyl butan-2-ol).

12. The process according to claim 1, wherein the reaction between the compound of formula (II) and sodium dicyanamide is performed at a concentration between 8 mL/g and 34 mL/g calculated from the amount of sodium dicyanamide.

13. The process according to claim 1, wherein the quantity of compound of formula (II) used in the reaction with sodium dicyanamide is between 1.9 and 3 equivalents calculated from the molar quantity of sodium dicyanamide.

14. The process according to claim 13, wherein the quantity of compound of formula (II) used in the reaction with sodium dicyanamide is between 1.9 and 2.5 equivalents calculated from the molar quantity of sodium dicyanamide.

15. The process according to claim 1, wherein the quantity of the base used to perform the reaction between the compound of formula (III) and methyl 6-chloropyridine-2-carboxylate is between 0.8 and 2 equivalents calculated from the molar quantity of compound of formula (III).

16. The process according to claim 15, wherein the quantity of the base used to perform the reaction between the compound of formula (III) and methyl 6-chloropyridine-2-carboxylate is between 1.5 and 2 equivalents calculated from the molar quantity of compound of formula (III).

17. The process according to claim 1, wherein the reaction between the compound of formula (III) and methyl 6-chloropyridine-2-carboxylate is performed at a concentration between 5 mL/g and 10 mL/g calculated from the amount of compound of formula (III).

18. The process according to claim 1, wherein the quantity of methyl 6-chloropyridine-2-carboxylate used in the reaction with compound of formula (III) is between 0.8 and 3 equivalents calculated from the molar quantity of compound of formula (III).

19. The process according to claim 18, wherein the quantity of methyl 6-chloropyridine-2-carboxylate used in the reaction with compound of formula (III) is between 1.5 and 2.5 equivalents calculated from the molar quantity of compound of formula (III).

20. The process according to claim 19, wherein the quantity of methyl 6-chloropyridine-2-carboxylate used in the reaction with compound of formula (III) is between 1.5 and 2.2 equivalents calculated from the molar quantity of compound of formula (III).

21. Use of N,N′-bis[(2R)-1,1,1-trifluoropropan-2-yl]triimidodicarbonic diamide hydrochloride of formula (III) for the preparation of vorasidenib.

22. The process according to claim 10, wherein the solvent used to perform the reaction between the compound of formula (II) and sodium dicyanamide is tert-amyl alcohol (i.e. 2-methyl butan-2-ol).

23. The process according to claim 10, wherein the reaction between the compound of formula (II) and sodium dicyanamide is performed at a concentration between 8 mL/g and 34 mL/g calculated from the amount of sodium dicyanamide.

24. The process according to claim 10, wherein the quantity of compound of formula (II) used in the reaction with sodium dicyanamide is between 1.9 and 3 equivalents calculated from the molar quantity of sodium dicyanamide.

25. The process according to claim 24, wherein the quantity of compound of formula (II) used in the reaction with sodium dicyanamide is between 1.9 and 2.5 equivalents calculated from the molar quantity of sodium dicyanamide.

26. The process according to claim 10, wherein the quantity of the base used to perform the reaction between the compound of formula (III) and methyl 6-chloropyridine-2-carboxylate is between 0.8 and 2 equivalents calculated from the molar quantity of compound of formula (III).

27. The process according to claim 26, wherein the quantity of the base used to perform the reaction between the compound of formula (III) and methyl 6-chloropyridine-2-carboxylate is between 1.5 and 2 equivalents calculated from the molar quantity of compound of formula (III).

28. The process according to claim 10, wherein the reaction between the compound of formula (III) and methyl 6-chloropyridine-2-carboxylate is performed at a concentration between 5 mL/g and 10 mL/g calculated from the amount of compound of formula (III).

29. The process according to claim 10, wherein the quantity of methyl 6-chloropyridine-2-carboxylate used in the reaction with compound of formula (III) is between 0.8 and 3 equivalents calculated from the molar quantity of compound of formula (III).

30. The process according to claim 29, wherein the quantity of methyl 6-chloropyridine-2-carboxylate used in the reaction with compound of formula (III) is between 1.5 and 2.5 equivalents calculated from the molar quantity of compound of formula (III).

31. The process according to claim 30, wherein the quantity of methyl 6-chloropyridine-2-carboxylate used in the reaction with compound of formula (III) is between 1.5 and 2.2 equivalents calculated from the molar quantity of compound of formula (III).
